(19) United States
(12) Patent Application Publication
(10) Pub. No.: US 2022/0411409 A1
(43) Pub. Date: Dec. 29, 2022

(54) PRODRUGS, ANALOGS OR DERIVATIVES OF ANTHOCYANIDIN AS ANTIVIRAL AGENTS

(71) Applicant: Sanctum Therapeutics Corporation, Sunnyvale, CA (US)

(72) Inventors: Parul P. Lakhlani, Sunnyvale, CA (US); Parthasarathy Krishnan, Sunnyvale, CA (US)

(21) Appl. No.: 17/806,990

(22) Filed: Jun. 15, 2022

Related U.S. Application Data
(60) Provisional application No. 63/202,530, filed on Jun. 15, 2021.

(51) Int. Cl. C07D 405/04; C07D 311/18; C07D 413/04; C07D 493/04

(52) U.S. Cl. CPC C07D 405/04; C07D 311/18; C07D 413/04; C07D 493/04

(57) ABSTRACT

The present invention relates to compounds which can inhibit host cell ACE2 receptor, viral spike S protein, Spike protein-ACE2 receptor interface, RNA-dependent RNA polymerase (RdRp), helicase and exoribonuclease activity. Particularly the invention relates to anthocyanidin analogs, derivatives and prodrugs as antiviral agents specific to Severe acute respiratory syndrome coronavirus 2 (SARS-CoV-2) or SARS-CoV-2 and SARS (Severe acute respiratory syndrome coronavirus).

### TECHNICAL FIELD

The present invention relates to the field of novel antiviral agents. Specifically, the present invention relates to compounds which can inhibit host cell ACE2 receptor, viral spike S protein, Spike protein-ACE2 receptor interface, RNA-dependent RNA polymerase (RdRp), helicase and exoribonuclease activity. Particularly the invention relates to anthocyanidin analogs, derivatives and prodrugs as antiviral agents specific to Severe acute respiratory syndrome coronavirus 2 (SARS-CoV-2) or SARS-CoV-2 and SARS (Severe acute respiratory syndrome coronavirus). Additionally, the present invention relates to compounds, compositions and methods for inhibiting SARS-CoV-2 viral replication, methods for treating or preventing the viral infection.

### BACKGROUND ART

An outbreak of pneumonia caused by severe acute respiratory syndrome coronavirus 2 (SARS-CoV-2) that started in Wuhan, China, at the end of 2019 and eventually led to a global pandemic. This virus seems more contagious than severe acute respiratory syndrome (SARS) coronavirus (SARS-CoV) and Middle East Respiratory Syndrome (MERS) coronavirus (MERS-CoV). Both SARS-CoV-2 and SARS-CoV enter host cells via the angiotensin-converting enzyme 2 (ACE2) receptor, which is expressed in various human organs. SARS-CoV-2 enters lung cells via the ACE2 receptor. The cell-free and macrophage-phagocytosed virus can spread to other organs and infect ACE2-expressing cells at local sites, causing multi-organ injury.

Entry into host cells is the first step of viral infection. A spike glycoprotein on the viral envelope of the coronavirus can bind to specific receptors on the membrane of host cells. Previous studies have shown that ACE2 is a specific functional receptor for SARS-CoV. Zhou et al. showed that SARS-CoV-2 can enter ACE2-expressing cells, but not cells without ACE2 or cells expressing other coronavirus receptors, such as aminopeptidase N and dipeptidyl peptidase 4 (DPP4), confirming that ACE2 is the cell receptor for SARS-CoV-2. Further studies showed that the binding affinity of the SARS-CoV-2 spike glycoprotein to ACE2 is 10- to 20-fold higher than that of SARS-CoV to ACE2. Because ACE2 is highly expressed in various organs and tissues, SARS-CoV-2 not only invades the lungs but also attacks other organs with high ACE2 expression. The pathogenesis of COVID-19 is highly complex, with multiple factors involved. The spike glycoprotein of SARS-CoV-2 is a potential target for the development of specific drugs, antibodies, and vaccines. Angiotensin-converting enzyme 2 (ACE2) is a membrane-bound zinc metallopeptidase that generates the vasodilatory peptide angiotensin 1-7 and thus performs a protective role in heart disease. It is considered an important therapeutic target in controlling the COVID-19 outbreak, since SARS-CoV-2 enters permissive cells via an ACE2-mediated mechanism.

EP2344154B1 The compositions include those that comprise anthocyanidin combinations rich in delphinidins, including delphinidins such as the ones found in berries. The compositions can optionally include either compositions that comprise andrographolides, such as the ones found in a plant of the genus Andrographis, or compositions that comprise combinations of myrtillin, quercetin, or caffeoyl quinic derivatives and proanthocyanidins, such as the ones found in the herba of a plant of the genus Vaccinium.

WO97/41137 A1 The invention relates to the use of an anthocyanidin or an anthocyanidin derivative of general formula (I) wherein R1, R2, R3 and R6 independently of each other is H, OH, alkoxy, an —O-glycosyl group, an —O-glycosyl group which is substituted with one or more acyl groups, or an —O-glycosyl moiety comprising at least two glycosyl groups and at least one acyl group arranged so that at least one acyl group is located between two glycosyl groups, R4 is OH, alkoxy, an —O-glycosyl group, an —O-glycosyl group which is substituted with one or one acyl groups, or an —O-glycosyl moiety comprising at least two glycosyl groups and at least one acyl group arranged so that at least one acyl group is located between two glycosyl groups, R5 is H, OH, and Y is a counterion or a salt, prodrug, a chemical modification or complex thereof for the preparation of a pharmaceutical composition for the prevention and/or treatment of neoplastic disorders.

EP0785790 A1 The invention relates to the use of an anthocyanidin or an anthocyanidin derivative of general formula (I) wherein R1, R2, R3 and R6 independently of each other is H, OH, alkoxy, an —O-glycosyl group, an —O-glycosyl group which is substituted with one or more acyl groups, or an —O-glycosyl moiety comprising at least two glycosyl groups and at least one acyl group arranged so that at least one acyl group is located between two glycosyl groups, R4 is OH, alkoxy, an —O-glycosyl group, an —O-glycosyl group which is substituted with one or more acyl groups, or an —O-glycosyl moiety comprising at least two glycosyl groups and at least one acyl group arranged so that at least one acyl group is located between two glycosyl groups, R5 is H, OH, and Y is a counterion or a salt, prodrug or complex thereof for the preparation of a pharmaceutical composition for the prevention and/or treatment of retroviral infections in mammals, as well as to novel anthocyanidin derivatives of general formula (I) and methods for preparation of said compounds and novel pharmaceutical compositions.

Accordingly, there is a need to develop drug compounds that can target the ACE2 receptor to provide treatment of viral infections from SARS-CoV, SARS-CoV-2 causing COVID 19 and SARS. Using docking and cell-based studies the present invention compounds which are anthocyanidin analogs that target ACE2 receptor (site for SARS-CoV and SARS-CoV-2 coronavirus entry into host cell, helicase, RdRp and exoribonucleases are disclosed.

### SUMMARY

It is primary object of the present invention to provide novel drug compounds for treatment of COVID 19 (severe acute respiratory syndrome coronavirus 2) and SARS severe acute respiratory syndrome.

It is another object of the present invention to provide novel drug compounds that are anthocyanidin analogs, derivatives or prodrugs that inhibit ACE2 receptor, RdRp, helicase and exoribonuclease activity and thus exhibits antiviral activity.

It is yet another object of the present invention to provide anthocyanidin analogs or anthocyanidin derivatives that inhibit the binding of Spike S-protein of SARS-CoV-2 with host ACE2 protein and reduce the entry of SARS-CoV-2 virus into host cells.

It is yet another object of the present invention to provide anthocyanidin analogs or anthocyanidin derivatives that inhibit the activity of exoribonuclease activity of nsp14.

It is yet another object of the present invention to provide anthocyanidin analogs or anthocyanidin derivatives that inhibit the activity of RNA-dependent RNA polymerase (RdRp).

It is another object of the present invention to provide a pharmaceutical composition comprising anthocyanidin analogs, derivatives or prodrugs.

It is another object of the present invention to provide an oral dosage form for administration of the drug for treatment of the viral diseases.

It is another object of the present invention to provide a method of treatment of viral diseases comprising administration of the pharmaceutical composition comprising the novel drug compounds of the present invention.

Thus according to the basic aspect of the present invention there is provided a compound represented by Formula (I) or a pharmaceutically acceptable salt thereof:

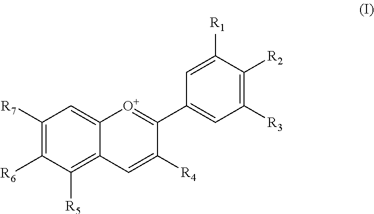

wherein the compound is anthocyanidin analog or anthocyanidin glycoside thereof, and wherein R₁ is selected from group comprising—hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

R₂ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

R1 and R2 are fused to form 1,3 dioxolane,

R₃ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

R₄ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

R₅ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

R₆ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

R₇ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

It is another aspect of the present invention to provide a compound represented by Formula (I) or a pharmaceutically acceptable salt thereof, wherein R₁-R₇ comprises halogen, —OH, —SH, —NH₂, —CN, —C(=0)OH, —C(=0)0(Ci-C₄ alkyl), —C(=0)NH₂, —C(=0)NH(C₁-C₄ alkyl), —C(=0)N(Ci-C₄ alkyl)₂, C1-C4 alkyl, —S(Ci-C₄ alkyl), C1-C4 alkoxy, C3-C6 cycloalkyl, C2-C5 heterocyclyl, —NH(C₁-C₄ alkyl), —N(C₁-C₄ alkyl)₂, phenyl, —C6H4OH, imidazole, and arginine.

It is another aspect of the present invention to provide a compound represented by Formula (I) or a pharmaceutically acceptable salt thereof, wherein at least one of R4, R5 or R7 is independently selected from —OR8, OR8R9R10, Wherein R8 is a glucose residue; and

wherein R8, R9 and R10 are independently selected from a glucose residue, a mannose residue, a galactose residue, a fucose residue, a rhamnose residue, an arabinose residue, xylose residue, a fructose residue, a glucuronic acid residue, and an apiose.

It is another aspect of the present invention to provide a compound represented by Formula (I) or a pharmaceutically acceptable salt as claimed in claim 1, wherein R₁ is —OH, R2 is —OCH3, R₃ is halogen, R₄ is Cl, R₅ is NO₂, R₆ is CN, and R₇ is NH2.

It is another aspect of the present invention to provide a pharmaceutical composition, comprising a compound of structure represented as Formula (I) and a pharmaceutically acceptable carrier or excipient.

| | | |
|---|---|---|
| 1 | 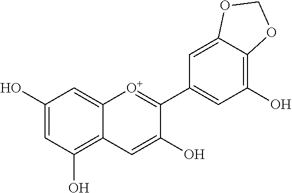 [SMILES: Oc1cc(O)c2cc(O)c(-c3cc(O)c4c(c3)OCO4)[o+]c2c1] | 3,5,7-trihydroxy-2-(7- hydroxybenzo[d][1,3]dioxol-5- yl)chromenylium |
| 2 | 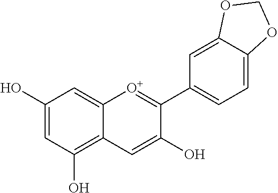 [SMILES: Oc1cc(O)c2cc(O)c(-c3ccc4c(c3)OCO4)[o+]c2c1] | 2-(benzo[d][1,3]dioxol-5-yl)- 3,5,7-trihydroxychromenylium |
| 3 | 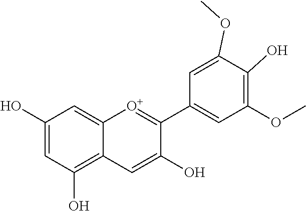 [SMILES: COc1cc(-c2[o+]c3cc(O)cc(O)c3cc2O)cc(OC)c1O] | 3,5,7-trihydroxy-2-(4-hydroxy- 3,5- dimethoxyphenyl)chromenylium |
| 4 | 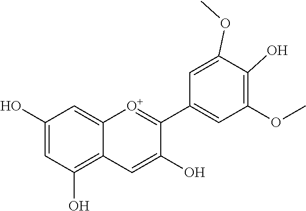 [SMILES: COc1cc(-c2[o+]c3cc(O)cc(O)c3cc2O)cc(OC)c1O] | 3,5,7-trihydroxy-2-(4-hydroxy- 3,5- dimethoxyphenyl)chromenylium |
| 5 | 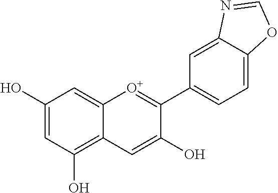 [SMILES: Oc1cc(O)c2cc(O)c(-c3ccc4ocnc4c3)[o+]c2c1] | 2-(benzo[d]oxazol-5-yl)-3,5,7- trihydroxychromenylium |
| 6 | 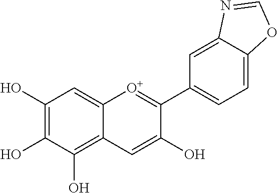 [SMILES: Oc1cc2c(O)c(O)c(O)cc2[o+]c1-c1ccc2ocnc2c1] | 2-(benzo[d]oxazol-5-yl)-3,5,6,7- tetrahydroxychromenylium |
| 7 | 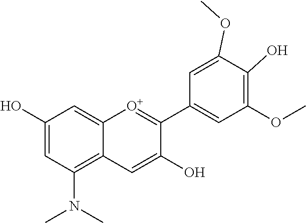 [SMILES: COc1cc(-c2[o+]c3cc(O)cc(N(C)C)c3cc2O)cc(OC)c1O] | 5-(dimethylamino)-3,7- dihydroxy-2-(4-hydroxy-3,5- dimethoxyphenyl)chromenylium |
| 8 | 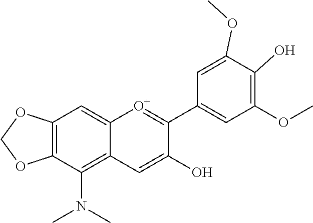 [SMILES: COc1cc(-c2[o+]c3cc4c(c(N(C)C)c3cc2O)OCO4)cc(OC)c1O] | 9-(dimethylamino)-7-hydroxy-6- (4-hydroxy-3,5- dimethoxyphenyl)- [1,3]dioxolo[4,5-g]chromen-5- ium |
| 9 | 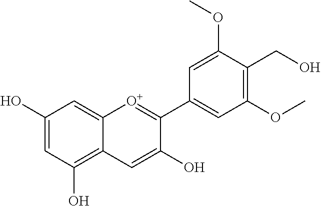 [SMILES: COc1cc(-c2[o+]c3cc(O)cc(O)c3cc2O)cc(OC)c1CO] | 3,5,7-trihydroxy-2-(4- (hydroxymethyl)-3,5- dimethoxyphenyl)chromenylium |
| 10 | 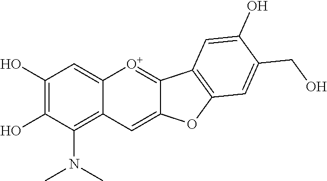 [SMILES: CN(C)c1c(O)c(O)cc2[o+]c3c(cc12)oc1cc(CO)c(O)cc13] | 1-(dimethylamino)-2,3,7- trihydroxy-8- (hydroxymethyl)benzofuro[3,2- b]chromen-5-ium |
| 11 | 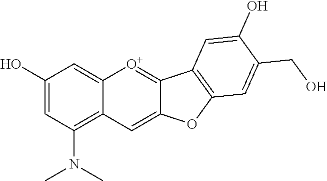 [SMILES: CN(C)c1cc(O)cc2[o+]c3c(cc12)oc1cc(CO)c(O)cc13] | 1-(dimethylamino)-3,7- dihydroxy-8- (hydroxymethyl)benzofuro[3,2- b]chromen-5-ium |
| 12 | 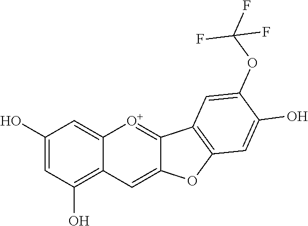 [SMILES: Oc1cc(O)c2cc3oc4cc(O)c(OC(F)(F)F)cc4c3[o+]c2c1] | 1,3,8-trihydroxy-7- (trifluoromethoxy)benzofuro[3,2- b]chromen-5-ium |
| 13 | 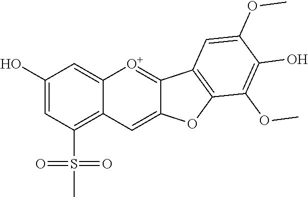 [SMILES: COc1cc2c(oc3cc4c(S(C)(=O)=O)cc(O)cc4[o+]c32)c(OC)c1O] | 3,8-dihydroxy-7,9-dimethoxy-1- (methylsulfonyl)benzofuro[3,2- b]chromen-5-ium |
| 14 | 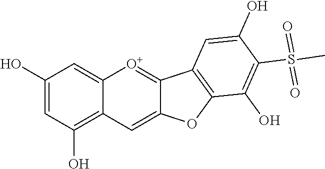 [SMILES: CS(=O)(=O)c1c(O)cc2c(oc3cc4c(O)cc(O)cc4[o+]c32)c1O] | 1,3,7,9-tetrahydroxy-8- (methylsulfonyl)benzofuro[3,2- b]chromen-5-ium |

It is another aspect of the present invention to provide a method of treating or preventing a viral infection in a subject in need thereof, comprising administering to the subject a therapeutically effective amount of a compound or a combination of compound of Formula (I), wherein the viral infection comprises infection by SARS-CoV or SARS-CoV2.

### DETAILED DESCRIPTION

The present invention discloses in various embodiments compounds with antiviral activity. In one embodiment is disclosed a compound of Formula (I), or its or a pharmaceutically acceptable salt thereof.

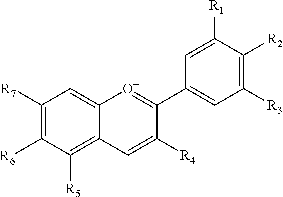

Wherein the compound is an anthocyanidin analog or an glycoside thereof. In another embodiment the compound may comprise of salts, derivatives, esters, ethers prodrugs and analogs of the compound of Formula (I)

In one embodiment R₁ is selected from group comprising—hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines

R₂ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines.

R₃ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines.

R₄ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines.

R₅ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines.

R₆ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines.

R₇ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines.

In another embodiment at least one of R4, R5 or R7 is independently selected from —OR8, OR8R9R10, Wherein R8 is a glucose residue, and R8, R9 and R10 are independently selected from a glucose residue, a mannose residue, a galactose residue, a fucose residue, a rhamnose residue, an arabinose residue, a xylose residue, a fructose residue, a glucuronic acid residue, and an apiose.

In a particular embodiment, the compound of Formula (I) comprises R₁ as —OH, R2 as —OCH3, R₃ as halogen, R₄ as Cl, R₅ as NO₂, R₆ as CN, and R₇ as NH2.

In another embodiment of the present invention the composition is a pharmaceutical composition of comprising one or more dosage forms. The pharmaceutical composition comprises the compound of Formula (I) and a pharmaceutically acceptable carriers or excipients. In a particular embodiment the pharmaceutical dosage form is a dosage form for oral administration. Particular embodiment the dosage form is selected from food or a drink for oral administration.

In another embodiment is disclosed a method of treating or preventing a viral infection in a subject in need thereof, comprising administering to the subject a therapeutically effective amount of a compound of Formula (I) or a combination of compounds as a dosage form, wherein the viral infection comprises infection by SARS-CoV or SARS-CoV2. The compounds represented by compound of Formula (I) show alleviation of viral diseases and improvement of symptoms of viral and infectious diseases, wherein the improvement in symptoms of viral and infectious diseases is due to modulation of ACE2 activity. Further the alleviation of symptoms of viral and infectious diseases is due to modulation of exoribonuclease, helicase and RdRp, RNA or DNA polymerase activity.

What is claimed is:

1. A compound represented by Formula (I) or a pharmaceutically acceptable salt thereof: 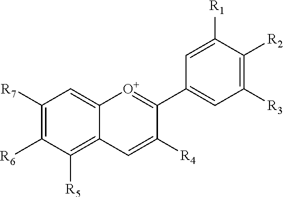
wherein the compound is anthocyanidin analog or a glycoside thereof, and
wherein R₁ is selected from group comprising—hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines;
R₂ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines;
R1 and R2 are fused to form 1,3 dioxolane,
R₃ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines;
R₄ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines;
R₅ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines;
R₆ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines;
R₇ is selected from group comprising hydrogen, alcohol, aryl, alkyl, alkoxy, acyl, alkenyl, alkynyl, heteroalkyl, heteroalkenyl, heteroalkynyl, carbocyclyl, heterocyclyl, hydroxyl, halogen, thiols, amides, amines.

2. The compound represented by Formula (I) or a pharmaceutically acceptable salt thereof as claimed in claim 1, wherein R₁-R₇ comprises halogen, —OH, —SH, —NH₂, —CN, —C(=0)OH, —C(=0)0(Ci-C₄ alkyl), —C(=0)NH₂, —C(=0)NH(C_{I}-C₄ alkyl), —C(=0)N(Ci-C₄ alkyl)₂, C1-C4 alkyl, —S(Ci-C₄ alkyl), C1-C4 alkoxy, C3-C6 cycloalkyl, C2-C5 heterocyclyl, —NH(C_{I}-C₄ alkyl), —N(C_{I}-C₄ alkyl)₂, phenyl, —C6H4OH, imidazole, and arginine.

3. The compound represented by Formula (I) or a pharmaceutically acceptable salt thereof as claimed in claim 1, wherein at least one of R4, R5 or R7 is independently selected from —OR8, OR8R9R10, Wherein R8 is a glucose residue, and
R8, R9 and R10 are independently selected from a glucose residue, a mannose residue, a galactose residue, a fucose residue, a rhamnose residue, an arabinose residue, a xylose residue, a fructose residue, a glucuronic acid residue, and an apiose.

4. The compound represented by Formula (I) or a pharmaceutically acceptable salt as claimed in claim 1, wherein R₁ is —OH, R2 is —OCH3, R₃ is halogen, R₄ is Cl, R₅ is NO₂, R₆ is CN, and R₇ is NH2.

5. The compound represented by Formula (I) or a pharmaceutically acceptable salt as claimed in claim 1, selected from the compounds set forth below or a pharmaceutically acceptable salt thereof:

| | | |
|---|---|---|
| 1 | 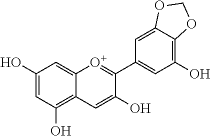 [SMILES: Oc1cc(O)c2cc(O)c(-c3cc(O)c4c(c3)OCO4)[o+]c2c1] | 3,5,7-trihydroxy-2-(7- hydroxybenzo[d][1,3] dioxol-5- yl)chromenylium |
| 2 | 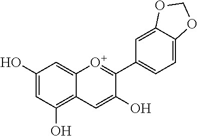 [SMILES: Oc1cc(O)c2cc(O)c(-c3ccc4c(c3)OCO4)[o+]c2c1] | 2-(benzo[d][1,3]dioxol- 5-yl)-3,5,7- trihydroxychromenylium |
| 3 | 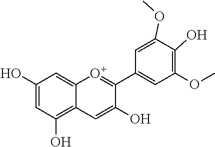 [SMILES: COc1cc(-c2[o+]c3cc(O)cc(O)c3cc2O)cc(OC)c1O] | 3,5,7-trihydroxy-2-(4- hydroxy-3,5- dimethoxyphenyl) chromenylium |
| 4 | 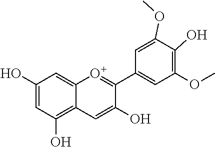 [SMILES: COc1cc(-c2[o+]c3cc(O)cc(O)c3cc2O)cc(OC)c1O] | 3,5,7-trihydroxy-2-(4- hydroxy-3,5- dimethoxyphenyl) chromenylium |
| 5 | 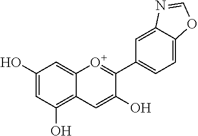 [SMILES: Oc1cc(O)c2cc(O)c(-c3ccc4ocnc4c3)[o+]c2c1] | 2-(benzo[d]oxazol-5- yl)-3,5,7- trihydroxychromenylium |
| 6 | 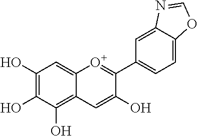 [SMILES: Oc1cc2c(O)c(O)c(O)cc2[o+]c1-c1ccc2ocnc2c1] | 2-(benzo[d]oxazol-5- yl)-3,5,6,7- tetrahydroxychromenylium |
| 7 | 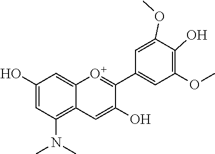 [SMILES: COc1cc(-c2[o+]c3cc(O)cc(N(C)C)c3cc2O)cc(OC)c1O] | 5-(dimethylamino)-3,7- dihydroxy-2-(4- hydroxy-3,5- dimethoxyphenyl) chromenylium |
| 8 | 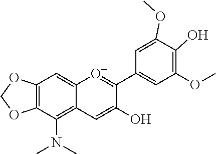 [SMILES: COc1cc(-c2[o+]c3cc4c(c(N(C)C)c3cc2O)OCO4)cc(OC)c1O] | 9-(dimethylamino)-7- hydroxy-6-(4-hydroxy- 3,5-dimethoxyphenyl)- [1,3]dioxolo[4,5-g] chromen-5-ium |
| 9 | 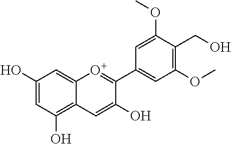 [SMILES: COc1cc(-c2[o+]c3cc(O)cc(O)c3cc2O)cc(OC)c1CO] | 3,5,7-trihydroxy-2-(4- (hydroxymethyl)-3,5- dimethoxyphenyl) chromenylium |
| 10 | 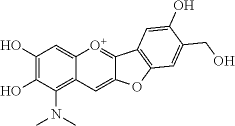 [SMILES: CN(C)c1c(O)c(O)cc2[o+]c3c(cc12)oc1cc(CO)c(O)cc13] | 1-(dimethylamino)- 2,3,7-trihydroxy-8- (hydroxymethyl) benzofuro[3,2-b] chromen-5-ium |
| 11 | 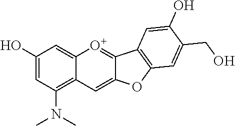 [SMILES: CN(C)c1cc(O)cc2[o+]c3c(cc12)oc1cc(CO)c(O)cc13] | 1-(dimethylamino)- 3,7-dihydroxy-8- (hydroxymethyl) benzofuro[3,2-b] chromen-5-ium |
| 12 | 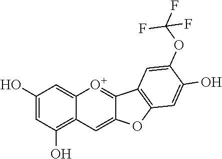 [SMILES: Oc1cc(O)c2cc3oc4cc(O)c(OC(F)(F)F)cc4c3[o+]c2c1] | 1,3,8-trihydroxy-7- (trifluoromethoxy) benzofuro[3,2- b]chromen-5-ium |
| 13 | 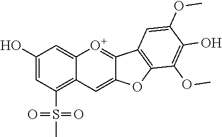 [SMILES: COc1cc2c(oc3cc4c(S(C)(=O)=O)cc(O)cc4[o+]c32)c(OC)c1O] | 3,8-dihydroxy-7,9- dimethoxy-1- (methylsulfonyl) benzofuro[3,2- b]chromen-5-ium |
| 14 | 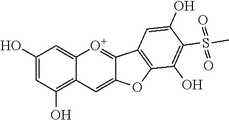 [SMILES: CS(=O)(=O)c1c(O)cc2c(oc3cc4c(O)cc(O)cc4[o+]c32)c1O] | 1,3,7,9-tetrahydroxy- 8-(methylsulfonyl) benzofuro[3,2- b]chromen-5-ium |

6. The pharmaceutical composition, comprising a compound as claimed in claim 1 and a pharmaceutically accept able carrier or excipient.

7. The pharmaceutical composition, comprising a compound as claimed in claim 1 wherein said composition is a food and drink.

8. A method of treating or preventing a viral infection in a subject in need thereof, comprising administering to the subject a therapeutically effective amount of a compound or a combination of compounds as claimed in claim 1, wherein the viral infection comprises infection by SARS-CoV or SARS-CoV2.
